(19) United States
(12) Patent Application Publication
(10) Pub. No.: US 2026/0157736 A1
(43) Pub. Date: Jun. 11, 2026

(54) SAMPLE COLLECTION DEVICE

(71) Applicant: Siro Diagnostics, Inc., San Diego, CA (US)

(72) Inventors: Cedric Bathany, Vista, CA (US); Michael David Jacobson, San Diego, CA (US); Clint Sever, San Diego, CA (US)

(21) Appl. No.: 19/126,577

(22) PCT Filed: Nov. 16, 2023
(86) PCT No.: PCT/US2023/079990

§ 371 Date: May 1, 2025

Related U.S. Application Data
(60) Provisional application No. 63/428,905, filed on Nov. 30, 2022.

(51) Int. Cl. A61B 10/00; B01L 3/00

(52) U.S. Cl. CPC A61B 10/0045; B01L 3/5088; B01L 2300/161

(57) ABSTRACT

Devices, systems, and methods for collecting samples for medical testing are described herein. The sample collection device includes a body portion extending along an axis between a first end to a second end opposite the first end and a tip portion between a third end and a fourth end opposite the third end, the fourth end being coupled to the body portion proximate the second end. The tip portion includes a fluid reservoir extending into the tip proximate the third end; and one or more lateral windows extending into the tip portion in a direction perpendicular to the axis such that a portion of the fluid reservoir is in fluid communication via the one or more windows.

### CROSS-REFERENCE TO RELATED PATENT APPLICATIONS

This application claims the benefit of and priority to U.S. Provisional Application No. 63/428,905, filed Nov. 30, 2022, which is hereby incorporated by reference in its entirety.

### TECHNOLOGICAL FIELD

The present technology relates generally to sample collection devices for collecting bio-fluidic samples for medical testing and diagnosis.

### BACKGROUND

Sample collection devices may be configured to collect cells and other biological material from various regions or locations. For example, sample collection devices may be used to collect samples such as blood, urine, saliva, or other fluids produced by the body.

### SUMMARY

According to various embodiments, a sample collection device for collecting a biological sample for analysis is provided. The sample collection device includes a body portion extending along an axis between a first end to a second end opposite the first end and a tip portion between a third end and a fourth end opposite the third end, the fourth end being coupled to the body portion proximate the second end. The tip portion includes a fluid reservoir extending into the tip portion proximate the third end and one or more lateral windows extending into the tip portion in a direction perpendicular to the axis such that a portion of the fluid reservoir is in fluid communication via the one or more windows.

The fluid reservoir may extend in a direction parallel to, or substantially parallel to, the axis from a first aperture proximate the third end to a second aperture. The second aperture may be in fluid communication with the one or more lateral windows. The first aperture may define a first cross-sectional area and the second aperture may define a second cross-sectional area that is smaller than the first cross-sectional area. The fluid reservoir may include a ledge surrounding a perimeter of the second aperture. The ledge may include a plurality of projections extending into the fluid reservoir. The first aperture may extend into the tip portion a first depth such that the fluid reservoir has a cross-sectional area from the first aperture to the first depth. The cross-sectional area may reduce along the axis such that the cross-sectional area is greater proximate the first aperture than the second aperture. The first aperture may be an oval shaped aperture defining a first axis and a second axis that is smaller than the first axis. The first axis may be greater than 0.8 mm and less than 1.6 mm and the second axis is between 0.6 mm and 1.4 mm. The first depth may be between 1.7 mm and 2.5 mm. A portion of the fluid reservoir may include a hydrophilic coating. Some or all of the fluid reservoir may include an anticoagulant coating.

The one or more lateral windows may include a first trapezoidal window having a first base edge proximate the second aperture and a second base edge opposite the first base edge, the second base edge being longer than the first base edge, and a second trapezoidal window having a third base edge proximate the second aperture and a fourth base edge opposite the first base edge, the third base edge being longer than the second base edge. The tip portion may be removably coupled to the body portion.

According to various embodiments, a system includes a cartridge including a cartridge aperture and a sample collection device for providing a sample to an interior of the cartridge. The sample collection device includes a tip portion configured to be at least partially received within the cartridge aperture. The tip portion includes a tip body extending between a first tip end and second tip end opposite the first tip end. The tip portion further includes a fluid reservoir configured to receive the sample and positioned between a first tip aperture proximate the first tip end and a second tip aperture, the first tip aperture defining a first cross-sectional area and the second tip aperture defining a second cross-sectional area smaller than the first cross-sectional area. The tip portion further includes one or more windows extending into the sample collection device such that the second tip aperture is in fluid communication with the one or more windows.

According to various embodiments, a sample collection device is configured to collect a sample. The sample collection device includes a sample collection body. The sample collection device includes a tip portion coupled to the sample collection body. The tip portion includes a fluid reservoir configured to receive the sample and positioned between a first tip aperture proximate a first tip end and a second tip aperture. The sample collection device further includes one or more windows extending into the sample collection device such that the first tip aperture and the second tip aperture is in fluid communication with the one or more windows.

### BRIEF DESCRIPTION OF THE DRAWINGS

Example embodiments are described below with reference to the accompanying drawings, wherein like numerals denote like elements. In the drawings:

FIGS. 1A-1B provide schematic depictions of an analyte detection system for analyzing the presence, absence, and/or quantity of one or more target analytes within a collected sample according to an example embodiment, wherein FIG. 1A shows components uncoupled and FIG. 1B shows components coupled for analysis and charging.

FIG. 1C provides a schematic depiction of another analyte detection system for analyzing the presence, absence, and/or quantity of one or more target analytes within a collected sample, wherein a charger is not provided, according to an example embodiment.

FIG. 2 illustrates perspective views of a sample collection device which is usable in the analyte detection systems of FIGS. 1A-1C, according to an example embodiment.

FIG. 3 illustrates a perspective view of the sample collection device, including a body portion and a tip portion, of FIG. 2.

FIG. 4 illustrates a partial perspective view of the body portion of FIG. 3.

FIG. 5 illustrates a perspective view of the tip portion of FIG. 3.

FIG. 6 illustrates another perspective view of the tip portion of FIG. 3.

FIG. 7 illustrates a side view of the tip portion of FIG. 3.

FIG. 8 illustrates a cross-sectional view of the tip portion of FIG. 3.

FIG. 9 illustrates another cross-sectional view of the tip portion of FIG. 3.

FIG. 10 illustrates a bottom view of the tip portion of FIG. 3.

FIG. 11 illustrates a cross-sectional perspective view of the tip portion of FIG. 3.

FIG. 12 illustrates a perspective view of another tip portion for use with the body portion of the sample collection device of FIG. 3, according to an example embodiment.

FIG. 13 illustrates a cross-sectional view of the tip portion of FIG. 12.

### DETAILED DESCRIPTION OF CERTAIN EMBODIMENTS

In the following detailed description, reference is made to the accompanying drawings, which form part of the present disclosure. The embodiments described in the drawings and description are intended to illustrate example embodiments and are not limiting. As used herein, the term “example” means “serving as an example or illustration” and should not necessarily be construed as preferred or advantageous over other embodiments. Other embodiments may be utilized, and modifications may be made without departing from the spirit or the scope of the subject matter presented herein. Aspects of the disclosure, as described and illustrated herein, can be arranged, combined, and designed in a variety of different configurations, all of which are explicitly contemplated and form part of this disclosure.

One aspect of the disclosure is directed to a system for detecting molecules. In various embodiments, the system includes a cartridge device, a reader device removably coupled to the cartridge device, and a sample collection device. The sample device is configured to be exposed to a sample for an analysis, such that the sample collection device receives a sample for medical testing. The sample collection device is further configured to be inserted into the cartridge device, which may be inserted into the reader device. The cartridge device and/or the reader device may cause medical testing to be performed on a portion of the sample collected by the sample collection device.

The sample collection device of various embodiments is configured to collect a sample from a specimen. Sample collection devices may be configured to collect cells and other biological material from any desired region or location, for example, a wound in the skin of a test subject. According to an example embodiment, the sample collection device includes a unit (e.g., a tip portion) that collects up to a predetermined volume of fluid (e.g., blood, urine, etc.) within a channel (e.g., fluid reservoir). For example, a volume of a fluid reservoir may define the predetermined volume. Further, an integrated stop (e.g., a capillary stop) at an end of the fluid reservoir may prevent over collection of the fluid within the channel. In other embodiments, the sample collection device may be configured to collect biological material, particulates, or other chemicals from the environment, such as, for example, from the air or the water, or from a physical surface or other structure.

The sample collection device of various embodiments is configured to collect a biological sample (e.g., blood, plasma, urine, etc.) for analysis in a detection system. The sample collection device includes a body portion extending along an axis between a first end to a second end opposite the first end. The sample collection device further includes a tip portion between a third end and a fourth end opposite the third end, the fourth end being coupled to the body portion proximate the second end. The tip portion includes a fluid reservoir extending into the tip portion proximate the third end. The fluid reservoir defines a predetermined volume configured to receive the sample. The fluid reservoir may include a stop (e.g., a capillary stop, an aperture, a cap, a limiter, etc.) configured to prevent the fluid reservoir from collecting more than the predetermined volume, thereby providing precise volume control for subsequent testing. The sample collection device may include one or more lateral windows extending into the tip portion in a direction perpendicular to the axis such that a portion of the fluid reservoir is in fluid communication via the one or more lateral windows. The one or more windows may be in fluid communication with the surrounding environment (e.g., open atmosphere), thereby placing the fluid reservoir in fluid communication with the atmosphere. In this sense, the one or more lateral windows may act as gas vents to the fluid reservoir to promote capillary flow of the sample into the fluid reservoir.

According to various embodiments, the fluid reservoir extends parallel, or substantially parallel, to the axis from a first aperture proximate the third end to a second aperture. According to various embodiments, at least a portion of the fluid reservoir is visible via a clear portion of the tip portion, thereby providing visual feedback to a user of the sample collection device. Thus, a portion of the fluid reservoir may be visible to a user of the fluid collection device such that the user may determine that the predetermined volume has been collected. For example, a user of the sample collection device may be able to see when the sample has reached to second aperture, thereby indicating that the desired volume has been collected. Further, a user of the sample collection device may be able to see that the fluid reservoir is not collecting a sample, which may indicate that the tip portion of the sample collection device needs to be repositioned.

The first aperture may define a first cross-sectional area and the second aperture may define a second cross-sectional area that is smaller than the first cross-sectional area. In this sense, the second aperture may serve as a stop (e.g., a capillary stop, a fluid flow stop, etc.) to reduce or prevent flow of the sample (e.g., via capillary action) through the fluid reservoir (e.g., preventing the sample from flowing out of the second aperture). The fluid reservoir may include a ledge surrounding a perimeter of the second aperture, the ledge including a plurality of projections surrounding the perimeter of the second aperture. The ledge and/or the steps may act as a stop to reduce or prevent capillary flow of the sample out of the second aperture.

The fluid reservoir may include a first aperture proximate the third end, the first aperture extending into the tip portion a first depth. The first aperture may extend into the tip portion a first depth such that the fluid reservoir has a cross-sectional area from the first aperture to the first depth. The cross-sectional area may reduce along the axis such that the cross-sectional area is greater proximate the first aperture than the second aperture. The first aperture may be an oval shaped aperture defining a first axis and a second axis that is smaller than the first axis. The oval shaped channel may facilitate capillary flow of the sample into the fluid reservoir. The first axis may, for example, be between 0.8 mm and 1.6 mm and the second axis may, for example, be between 0.6 mm and 1.4 mm. In more particular embodiments, the first axis may be greater than 1 mm and less than 1.3 mm and the second axis may be between 0.7 mm and 1 mm.

The first axis (e.g., a major axis) and the second axis (e.g., a minor axis) may be customized based on the target sample fluid. For example, certain samples may have differing fluidic properties, and therefore, capillary flow may be improved by altering the dimensions of the first aperture and/or the fluid reservoir. Further, the dimensions of the first aperture and/or the fluid reservoir may be adjusted based on a desired volume (e.g., predetermined volume) of a sample to be collected. The depth may be customized to collect a desired volume of the sample. For example, in particular implementations the first depth may be between 1.7 mm and 2.5 mm. The predetermined volume may, in particular implementations, be between 1 uL and 5 ul or more particularly between 2 uL and 3 uL.

A portion of the fluid reservoir may include a hydrophilic coating which may facilitate capillary flow of the sample in the fluid reservoir and/or releasing the sample from the fluid reservoir for testing. Additionally, or alternatively, a portion of the fluid reservoir may include an anticoagulant coating which may facilitate capillary flow of the sample in the fluid reservoir, preserving the sample within the fluid reservoir, and/or releasing the sample from the fluid reservoir for testing.

According to various embodiments, the fluid reservoir 812 includes a coating that has both anticoagulant and hydrophilic properties. In this sense, a single coating may be applied to some or all of the fluid reservoir 812 to achieve both anticoagulant and hydrophilic properties. For example, the coating may one or more of the following components: Polyvinylpyrrolidone (PVP), Polyethylene glycol (PEG), water (e.g. deionized water (DI)), and/or Ethylenediaminetetraacetic acid (EDTA). The coating may be prepared as a solution before applying the coating to the fluid reservoir 812. For example, per 10 mL of coating, the coating may include between 50-75 mg PVP (e.g., weight per volume), 250-300 mg PEG (e.g., weight per volume), 1 Ml of DI (e.g., volume per volume), and/or 9 Ml of EDTA (e.g., volume per volume). The PVP may have a molecular weight (MW) of about 40,000 (e.g., between 30,000 MW and 50,000 MW). The PEG may include 35 kilo-Dalton (kDa) PEG at a 25% weight ratio. The EDTA may include a 0.5 millimole/milliliter (e.g., 500 Millimolar (mM)) EDTA having a pH of about 8.0.

The one or more lateral windows may include a first geometrical (e.g., trapezoidal, rectangular, triangular, circular, oval, etc.) window having a first base edge proximate the second aperture and a second base edge opposite the first base edge, the second base edge being longer than the first base edge and a second trapezoidal window having a third base edge proximate the second aperture and a fourth base edge opposite the first base edge, the third base edge being longer than the second base edge. Arranging the lateral windows as such may provide additional strength by providing angled supports on the sides of the lateral windows.

The tip portion may be removably coupled to the body portion. In this sense, the tip portion may be removed for further processing (e.g., testing) of the sample collected within the fluid reservoir. Further, a new tip portion may be used each time the sample collection device is used to collect a sample, which may allow the sample collection device to be reused and/or used by multiple users.

The sample collection device of various embodiments described herein is sized and shaped to collect a sufficiently large sample from an appropriate location of a specimen such that it is possible, using the other devices described below, to detect the presence, absence, and/or quantity of one or more target analytes in and/or on the specimen. For example, the sample collection device may be appropriate a blood sample for detection of one or more analytes in blood and/or any type of sample. While reference to collecting blood is discussed in detail herein, it should be appreciated that the sample collection devices described herein may be configured to collect other bodily fluids (e.g., urine, saliva, etc.) and may be appropriate for collecting target analytes for various tests, including, for example, tests for tracking fertility, pregnancy, drug levels, sexual health markers, and/or other various analytes.

The sample collection device of various embodiments is configured to collect a sample from a specimen. The sample collection device may be configured to collect a blood sample. For example, a user of the sample collection device may create a wound (e.g., prick a finger) the skin of a test subject and the sample collection device may be used to collect a controlled volume (e.g., a predetermined volume) of blood from the wound.

In various embodiments, a cartridge is formed of a housing, which defines an enclosed space and has various features that enable the cartridge to do one or more of the following: receive a sample with target analytes from a sample collection device, store the sample with sample preparation reagents, provide a space for mixing and binding of the target analytes with sample preparation reagents, provide an analysis zone wherein bound target analytes localize over sensors for detection, provide a fluid medium for transporting the bound target analytes to the analysis zone, store and provide a substrate that can undergo a detectable reaction when introduced to the bound target analytes, provide a fluid medium for transporting the substrate to the bound target analytes in the analysis zone, and provide a waste collection zone where waste is stored.

In various embodiments, the cartridge is a substantially closed system wherein the reactions needed to detect the presence, absence, and/or quantity of one or more target analytes occur within the cartridge. The cartridge of such embodiments is said to be “substantially closed” because the only inputs needed into the cartridge system are one or more of the following: a sample from a specimen, energy to facilitate mixing and binding, and a magnetic force to facilitate localization of bound target analytes within an analysis zone; the only outputs from the cartridge are electrical signals. The cartridge may be a target analyte-specific with the included sample preparation reagents selected to detect one or more specific target analytes. Different cartridge types include different reagents intended to identify different target analytes. For example, different cartridge types may include inflammation, influenza, COVID-19, testosterone, fertility, HIV, and Vitamin D, which each include application-specific reagents intended to identify different target analytes.

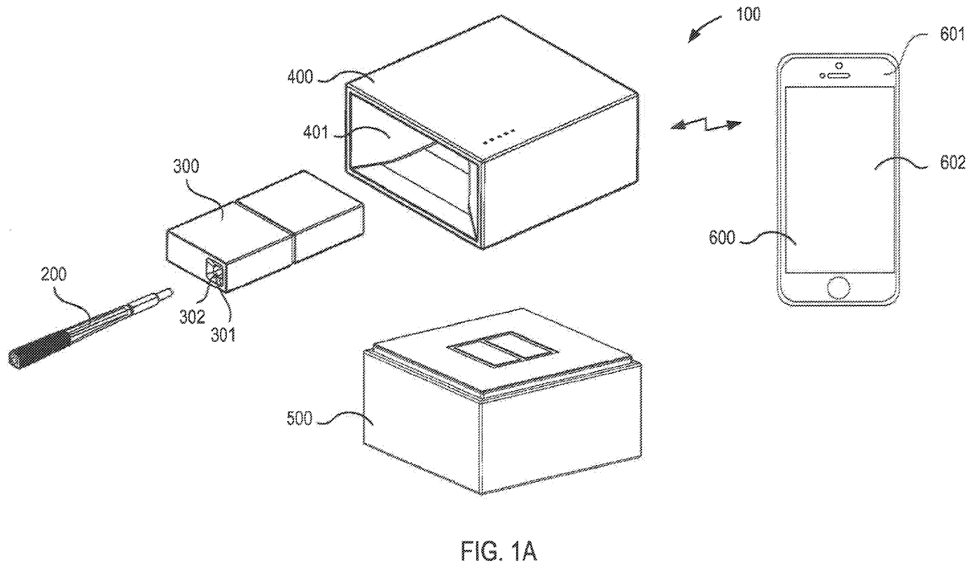

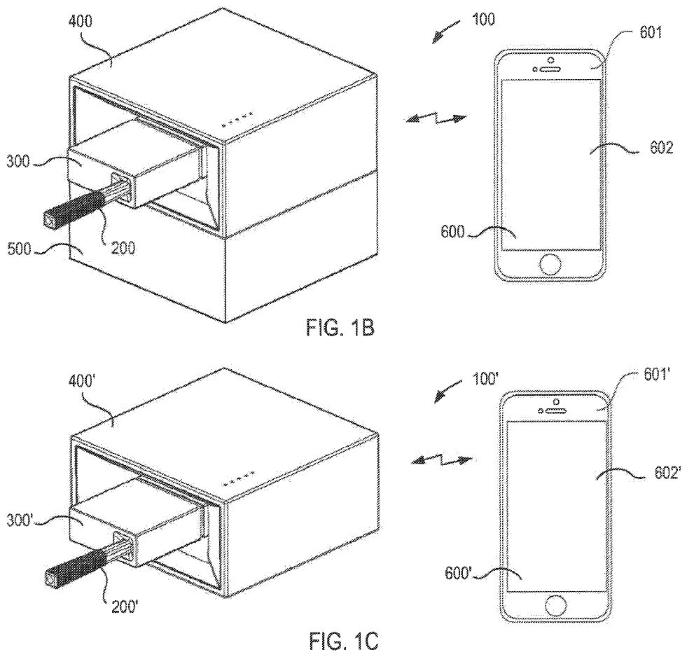

Referring now to FIGS. 1A and 1B, an analyte detection system 100 is shown, according to an example embodiment. The detection system 100 may include a sample collection device 200, a cartridge device 300, a reader device 400, a charger 500, and/or a software-based detection interface system 600. The detection system 100 may be used to detect the presence, absence, and/or quantity of one or more target analytes.

The sample collection device 200 is configured to be exposed to a sample for analysis. For example, the sample collection device 200 may be exposed to a biological sample, such as, but not limited to, blood for determining the presence, absence, and/or quantity of one or more target analytes within the sample.

The cartridge device 300 is configured to analyze the sample collected with the sample collection device 200. The cartridge device 300 may include an input tunnel 301 that extends from an aperture 302 into the cartridge housing. The input tunnel 301 is configured to permit insertion of the sample collection device 200 as shown in FIG. 1B such that the collected sample may be analyzed within the cartridge device 300. Based on the analysis, the cartridge device 300 is configured to generate electric signals indicative of the presence, absence, and/or quantity of one or more target analytes within the sample.

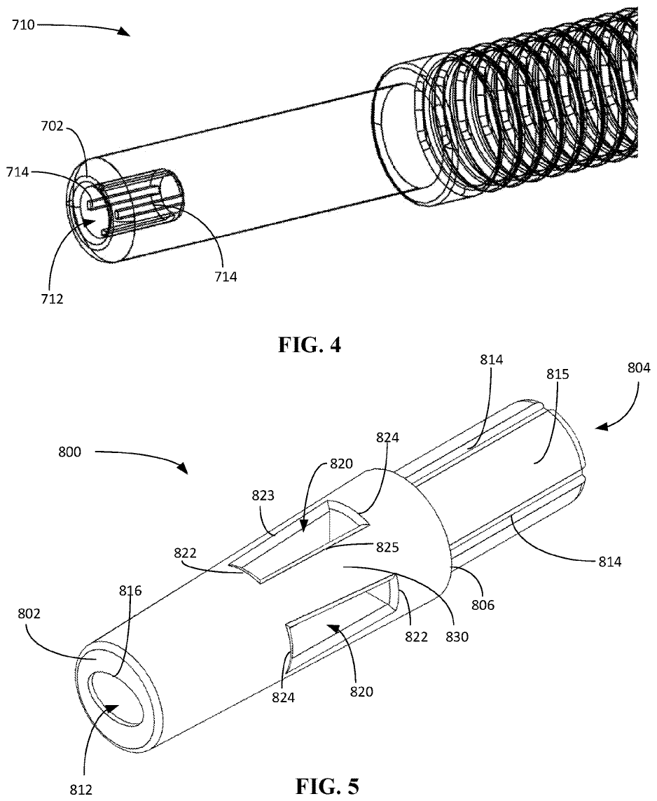

The reader 400 is configured for electric coupling with the cartridge device 300 to permit transmission of the electric signals indicative of the presence, absence, and/or quantity of one or more target analytes within the sample generated by the cartridge device 300. The cartridge device 300 may be electrically coupled to the reader 400 by inserting the cartridge device 300 within a reader opening 401 of the reader 400, as shown in FIG. 1B, such that the respective electrical connectors of the cartridge device 300 and the reader 400 contact one another. The reader 400 may comprise a computer readable medium with instructions that, when executed by a processor of the reader 400, cause electrical components of the cartridge device 300 to perform steps for analyzing the sample on the sample collection device 200. The instructions may not be executed until the cartridge device 300 is electrically coupled to the reader 400 and the sample collection device 200 is suitably disposed within the cartridge device 300, for example, as shown in FIGS. 1B or 4A.

In one embodiment, a portion of the sample collection device 200 (e.g., the tip portion) and the cartridge device 300 are each disposable and designed for one time use while the reader 400 is designed for multi-use and for receiving many different cartridge devices throughout the life of the reader 400 such that many samples are analyzed by the reader 400 for determining the presence, absence, and/or quantity of one or more target analytes within the respective samples. Such a configuration is expected to promote sanitary use of the system, as the components exposed to the sample are disposable, while reducing costs as the components with more expensive electronics (e.g., the reader 400), may be used repeatedly.

The charger 500 is configured to charge one or more batteries within the reader 400, e.g., via respective inductive coils disposed within the housings of the charger 500 and the reader 400. The charger 500 may be plugged into a conventional socket, e.g., via a cord or a cord with an AC to DC power converter, for charging components within the charger 500 to permit charging of the reader 400.

As will be readily apparent to one skilled in the art, the detection system need not require a charger. For example, referring to FIG. 1C, a detection system 100′ is constructed similarly to the detection system 100 of FIGS. 1A and 1B, wherein like components are identified by like-primed reference numbers. Thus, for example, a cartridge device 300′ in FIG. 1C corresponds to the cartridge device 300 of FIGS. 1A and 1B, etc. As will be observed by comparing FIGS. 1C and 1B, the detection system 100′ does not include the charger 500. In such an embodiment, the reader 400′ may be plugged into a conventional socket, e.g., via a cord or a cord with an AC to DC power converter, for powering components of the reader 400′ and/or the reader 400′ may include a suitable battery such as a replaceable battery or rechargeable battery and the reader 400′ may include circuitry for charging the rechargeable battery, and a detachable power cord.

In FIGS. 1A and 1B, a software-based detection interface system 600 is installed and runs on a computing device 601 to permit a user to review analyte detection test results, e.g., on a display 602 of a computing device 601, according to various example embodiments. The computing device 601 may be, for example, a smartphone, smartwatch, tablet, wearable device, a laptop or other computer. As shown in FIGS. 1A and 1B, the reader 400 may communicate with the computing device 601 wirelessly to transmit data indicative of the presence, absence, and/or quantity of one or more target analytes based on the electrical signals generated within the cartridge device 300. In addition, or alternatively, a removable wired connection, such as a cable connection, may be provided between the reader 400 and the computing device 601. The software-based detection interface system 600 may comprise a computer readable medium with instructions that, when executed by a processor of the computing device 601, cause the display 602 to display information indicative of the presence, absence, and/or quantity of one or more target analytes. It should be appreciated that, the software-based detection interface system 600 may be omitted from the detection system 100. For example, the reader device 400 may include a display or other visual indication system that provides results and/or other information to a user of the detection system 100 without the need for an external computing device 601 and/or a software-based detection interface system 600.

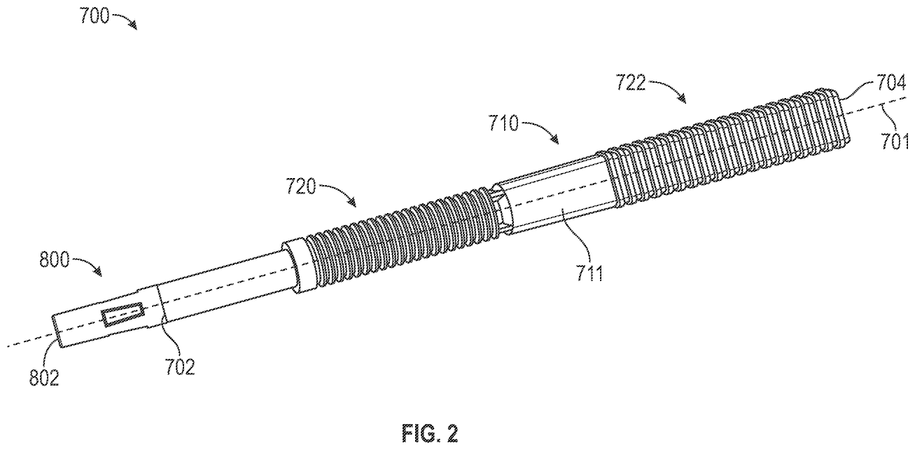

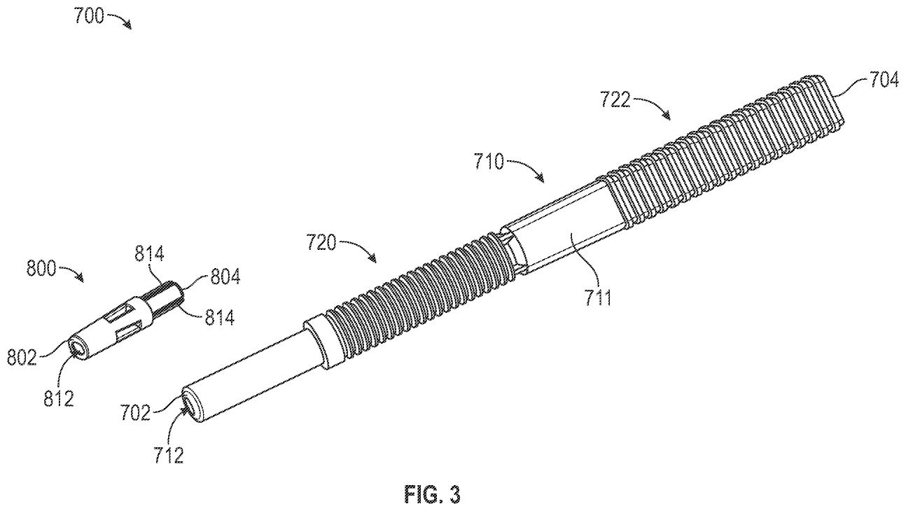

Referring now to FIGS. 2 and 3, perspective views of an example sample collection device 700, including a body portion 710 and a tip portion 800, which is usable in the analyte detection system 100 of FIGS. 1A-1C, is shown. The sample collection device 700 may share one or more features with any of the other sample collection devices described herein (e.g., the sample collection device 200). For example, the sample collection device 700 is configured to collect a small quantity of a sample (e.g., blood) to be analyzed and configured for full or partial insertion within the cartridge device 300 after sample collection.

The sample collection device 700 is configured to collect a predetermined volume of a sample. The predetermined volume may correspond with a volume of a fluid reservoir 812 (e.g., a collection channel, a collection tube, a metered capillary tube, a metered capillary channel, etc.) positioned within a tip portion 800 of the sample collection device 700. The fluid reservoir 812 may be used to collect a predetermined volume of a sample (e.g., blood) for insertion within the cartridge device 300 where various testing may be performed. Collection of a predetermined volume of the sample may promote accuracy of analyte analysis as a substantially known quantity of the sample will be analyzed.

The sample collection device 700 extends along an axis 701 between a second end 704 of a body portion 710 and a third end 802 of a tip portion 800. In a particular embodiment, the tip portion 800 may be removably coupled to the body portion 710 such that the tip portion 800 may be removed from the body portion 710, as shown in FIG. 3. For example, the body portion 710 includes an aperture 712 proximate the first end 702 configured to receive at least a portion of a projection 815 of the tip portion 800. As is discussed further herein, the projection 815 defines a plurality of grooves 814 configured to receive projections within the aperture 712 (e.g., the projections 714 shown in FIG. 4) to stabilize the tip portion 800 with respect to the body portion 710 (e.g., by preventing relative rotation between the body portion 710 and the tip portion 800). Alternatively, the tip portion 800 may be non-removably secured to the body portion 710.

According to various embodiments, the tip portion 800 may be intended for single use. For example, the tip portion 800 may be removed and exchanged for a new tip portion 800 after collecting a sample of blood. In this sense, the tip portion 800 can be discarded and replaced between uses, rather than cleaning and/or sterilizing. For example, multiple users may utilize the same body portion 710 by using a new tip portion 800 with each sample collected.

The body portion 710 extends from a first end 702 to the second end 704. The body portion 710 includes a handle 711 sized and shaped to be held by a collector's hand. The handle 711 may include gripping protrusions 720, 722 as illustrated. The handle 711 may further the lock the sample collection device 200 within the input tunnel of cartridge device 300.

The tip portion 800 extends from a third end 802 to a fourth end 804. The tip portion 800, is configured to be exposed to a sample such that, at most, a predetermined volume of the sample is disposed within the fluid reservoir 812 for analysis. Collection of a predetermined volume of the sample is expected to promote accuracy of analyte analysis as a substantially known quantity of the sample will be analyzed. The tip portion 810 may be transparent to permit a collector to verify that sample is disposed in the tube fluid reservoir. The tip portion 800 may have a rounded end (e.g., proximate the third end 802) as illustrated although various shapes may be used including any blunt or substantially blunt tip shape. Further, the tip may not be blunt. For example, the tip may include a sharp edge that surrounds a first aperture. The tip may be generally any geometrical shape (e.g., rectangle, oval, circle, etc.). The tip portion 800 may be configured to collect a sample from any desired region or location, for example, from a wound created in the skin on a finger, or from another body part.

Referring now to FIG. 4, a partial perspective view of the first end 702 of the body portion 710 is shown, according to an example embodiment. The body portion 710 includes an aperture 712 configured to receive a portion of the tip portion 800. Further, the body portion 710 includes a plurality of projections 714 positioned within aperture 712. The projections 714 are positioned radially about the aperture 712 such that the projections 714 are received within the respective grooves 814 in the tip portion 800 (see FIG. 5) to stabilize the tip portion 800 with respect to the body portion 710 (e.g., by preventing relative rotation between the body portion 710 and the tip portion 800). It should be appreciated that, according to other embodiments, the tip portion 800 may include projections that are received within grooves in the body portion 710 in a similar manner.

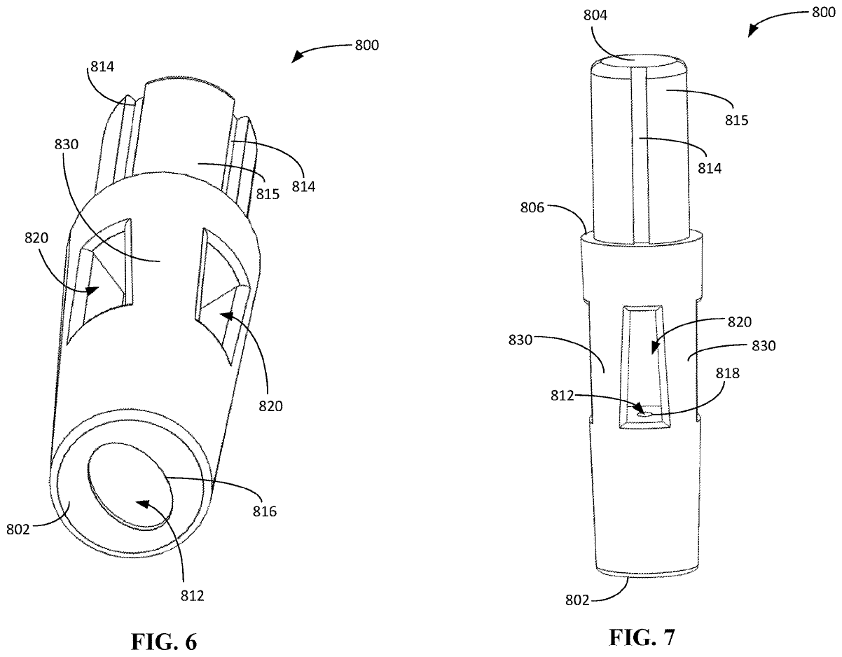

Referring now to FIGS. 5 and 6, perspective views of the tip portion 800 are shown, according to various embodiments. The tip portion 800 includes a plurality of grooves 814 that surround a portion of the tip portion 800 proximate the fourth end 804 (e.g., the portion that is received within the aperture 712 of the body portion 710). The tip portion 800 further includes a shoulder 806. The shoulder 806 may prevent the tip portion 800 from translating within the 712 beyond a desired distance. For example, the shoulder 806 may interface with the first end 702 of the body portion 710 to prevent the tip portion 800 from overextending into the aperture 712.

The tip portion 800 includes a fluid reservoir 812 proximate the third end 802. The fluid reservoir 812 may be configured to receive a predetermined volume of a sample (e.g., blood). The fluid reservoir 812 extends between a first aperture 816 and a second aperture 818 (see FIG. 7), such that the predetermined volume of the sample is received between the first aperture 816 and the second aperture 818.

The tip portion 800 includes a plurality of windows 820 (e.g., lateral windows) that surround (e.g., laterally surrounding) a portion of the tip portion 800. The windows 820 are in fluid communication with the fluid reservoir 812 and configured to promote gas flow (e.g., air flow) from the fluid reservoir 812 out of the tip portion 800 (e.g., via the second aperture 818 shown in FIG. 7). Further, the windows 820 may be configured to expose a portion of the fluid reservoir 812, as is discussed further with respect to FIG. 7. The windows 820 extend into the tip portion 800 in a direction perpendicular to the axis 701 (see FIG. 2). The windows 820 are defined by a first base 824 connected to a second base 822 via a first leg 823 and a second leg 825. The first base 824 is longer than the second base 822. Further, the windows 820 are arranged such that the first base 824 alternates sides (e.g., between proximate the third end 802 and proximate the fourth end 804), thereby creating an angled support 830 between each of the respective windows 820. The angled support 830 may provide structural strength and rigidity as opposed to a support 830 that is parallel to the axis 701 (see FIG. 2) or substantially parallel to (e.g., +/−10 degrees) the axis 701.

Referring now to FIG. 7, a side view of the tip portion 800 is shown, according do an example embodiment. The second aperture 818 defines an end of the fluid reservoir 812. Thus, in use, the sample is received within the fluid reservoir 812 between the first aperture 816 and the second aperture 818. According to various embodiments, some or all of the tip portion 800 between the first aperture 816 and the second aperture 818 may be transparent such that a collector (e.g., a user of the sample collection device 700) may be able to see when a sample (e.g., blood) is received within the fluid reservoir 812. For example, some or all of the tip portion 800 may be made from a clear plastic material, a glass material, etc. A sample being visible via the clear portion of the tip portion 800 may provide an indication that a sufficient volume (e.g., a predetermined volume) of the sample has been collected.

Further, a user of the sample collection device 700 may be able to see if the fluid reservoir 812 is not collecting a sample (e.g., as indicated by the lack of a sample visible via the clear portion), which may indicate that the tip portion 800 of the sample collection device 700 should be repositioned to collect the sample.

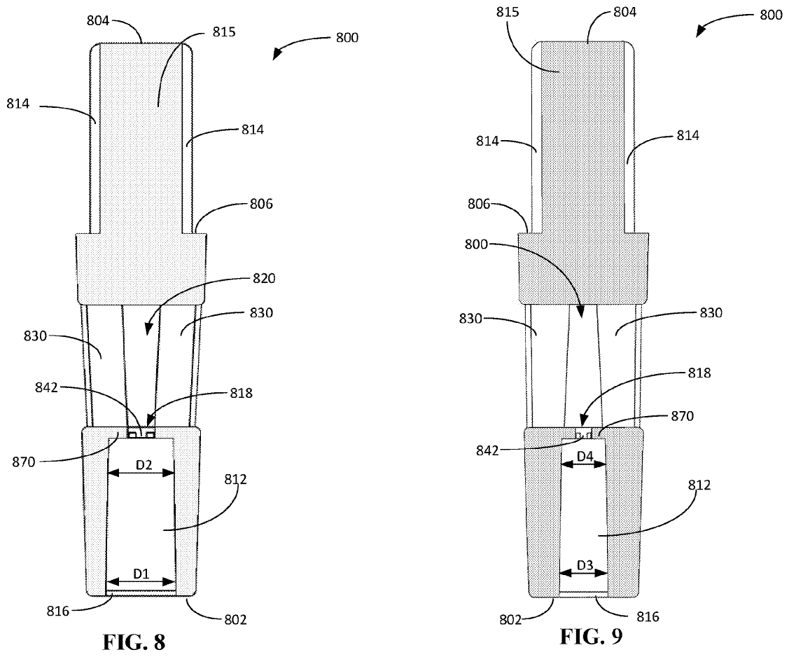

Referring now to FIGS. 8 and 9, cross-sectional views of the tip portion 800 are shown, according to an example embodiment. The tip portion 800 includes a fluid reservoir 812 configured to receive a sample (e.g., for use in conjunction with the detection system 100 described above). The fluid reservoir 812 is configured to promote flow of the sample (e.g., blood flow) from the first aperture 816 to the second aperture 818. For example, in use, a wound may be created in the skin and the third end 802 of the tip portion 800 may be positioned on top of the wound such that blood flows from the wound and into the fluid reservoir 812.

As described above, some or all of the fluid reservoir 812 (e.g., a portion of the fluid reservoir 812) may be coated in a hydrophilic coating, which may promote spontaneous wicking (e.g., capillary flow). Additionally or alternatively, at least a portion of the fluid reservoir 812 may be coated in an anticoagulant coating, which may promote spontaneous wicking (e.g., capillary flow) of certain samples (e.g., blood). The coating may cause the fluid reservoir 812 (e.g., the sidewalls, the plurality of projections 842, and/or the ledge 870) to have both hydrophilic and anticoagulant properties.

According to various embodiments, the fluid reservoir 812 is dimensioned to promote spontaneous wicking (e.g., capillary flow). For example, the cross-sectional area the fluid reservoir 812 may be less than 2 mm². The cross-sectional area of the fluid reservoir may be greater than 0.5 mm². When the third end 802 is applied to a sample collection site, the fluid reservoir 812 may cause spontaneous wicking of the sample into the fluid reservoir 812. For example, a sample may be drawn into the fluid reservoir 812 via the first aperture 816 and air within the fluid reservoir 812 may be expelled via second aperture 818 and the windows 820 until the sample reaches a stop in the fluid reservoir 812.

According to various embodiments, the fluid reservoir 812 includes a stop proximate the second aperture 818. The stop includes the ledge 870 (e.g., a rim, an edge, etc.) that defines the second aperture 818. The ledge 870 creates a sudden decrease in the cross-sectional area of the fluid reservoir 812, which may hinder or completely stop the spontaneous wicking of the sample. Thus, the ledge 870 may help prevent collection of a sample that is greater than a desired volume (e.g., a predetermined volume). The second aperture 818 may define a depth of the fluid reservoir (e.g., the distance between the first aperture 816 and the second aperture 818). For example, the sample may flow past the ledge 870 but stop before exiting the second aperture 818. The depth may be customized based on the desired volume (e.g., the predetermined volume) of the sample to be collected. The depth may be between is between 1.7 mm and 2.5 mm.

According to various embodiments, the stop includes a smaller cross-sectional area of the fluid reservoir 812 proximate the second aperture 818. For example, The cross-sectional area of the fluid reservoir 812 decreases from the first aperture 816 until the ledge 870 such that D1 is greater than D2 and D3 is greater than D4. The reduction in cross-sectional area may increase the speed of the spontaneous wicking, thereby reducing the collection period required to collect the predetermined volume.

According to various embodiments, the stop includes a change in geometry of the cross-sectional area of the fluid reservoir 812 (e.g., a change in surface topography) proximate the second aperture 818. For example, a sudden change in the cross-sectional area may result in a sharp edge within the fluid reservoir 812 (e.g., a sharp edge extending along the axis 701 shown in FIG. 2) to slow or stop the spontaneous wicking of the sample into the fluid reservoir 812.

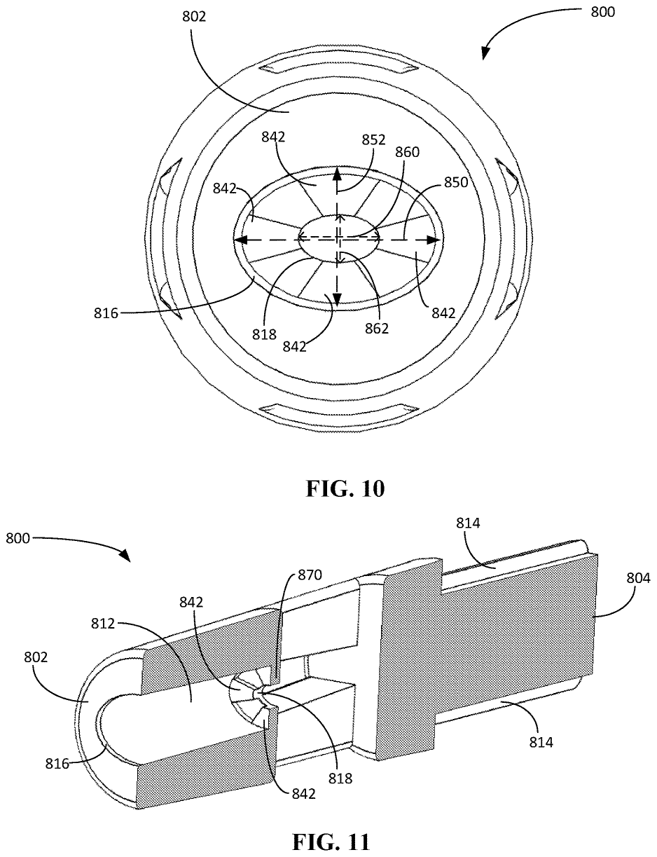

As is discussed below with respect to FIGS. 10 and 11, the change in geometry of the cross-sectional area (e.g., a change in surface topography) may slow or stop spontaneous wicking. The change in geometry of the cross-sectional area (e.g., a change in surface topography) includes a plurality of projections 842 surrounding the second aperture 818. The plurality of projections 842 create a local change in geometry (e.g., a change in the cross section), which causes a change in the contact angle of the sample as the sample spontaneously wicks into the fluid reservoir 812. The change in contact angle may require additional pressure to push the sample past the change in geometry of the cross-sectional area (e.g., a change in surface topography) and/or past the second aperture 818. The fluid reservoir 812 may not generate sufficient pressure to overcome pressure required to propel the sample past the change in geometry of the cross-sectional area (e.g., a change in surface topography), thereby stopping or otherwise inhibiting flow of the sample before the sample flows past the second aperture such that a controlled volume (e.g., a predetermined volume) of the sample is collected.

Referring now to FIG. 10, a bottom view of the tip portion 900 is shown, according to an example embodiment. The first aperture 816 is generally oval shaped and defines a first axis 850 and a second axis 852 that is smaller than the first axis 850. An oval shaped aperture 816 and fluid reservoir 812 may facilitate spontaneous wicking (e.g., increase speed of the wicking to reduce time required to collect a sample) of the sample into the fluid reservoir 812 (e.g., by increasing the surface area per volume ratio). An oval shaped aperture 816 and fluid reservoir 812 may result in a sufficiently large volume of sample being collected. It should be appreciated that, according to other embodiments, the first aperture 816, the fluid reservoir 812, and/or the second aperture 818 may be circular shaped.

According to various embodiments, the first axis 850 is between 0.3 mm and 2 mm. The first axis 850 may be between 0.7 mm and 1.5 mm. The first axis 850 may be between 1 mm and 1.3 mm. The second axis 852 may be between 0.3 mm and 2 mm. The second axis 852 may be between 0.5 mm and 1.2 mm. The second axis 852 may be between 0.7 mm and 1 mm.

The second aperture 818 is generally oval shaped and defines a first axis 860 and a second axis 862 that is smaller than the first axis 860. The second aperture 818 is concentric with the first aperture 816. The cross-sectional area of the second aperture 818 is smaller than the first aperture 816, which may reduce or stop spontaneous wicking of the sample as the sample approaches the second aperture 818.

According to various embodiments, the first axis 860 is between 0.3 mm and 2 mm. The first axis 860 may be between 0.5 mm and 1.2 mm. The first axis 860 may be between 0.7 mm and 1 mm.

Referring now to FIG. 11, a cross-sectional perspective view of the tip portion 800 is shown, according to an example embodiment. The ledge 870 includes a change in geometry of the cross-sectional area of the fluid reservoir 812 (e.g., a change in surface topography) (e.g., the plurality of projections 842) proximate the second aperture 818. The change in geometry of the cross-sectional area (e.g., a change in surface topography) may slow or stop spontaneous wicking by disrupting the flow of the sample through the fluid reservoir. The change in geometry (e.g., a change in surface topography) includes a plurality of projections 842 surrounding the second aperture 818. The plurality of projections 842 create a plurality of steps that surround the second aperture 818. Each step defines one or more sharp edges (e.g., extending along the axis 701 shown in FIG. 2). The sharp edges create a change in the cross-sectional area of the fluid reservoir 812 (e.g., with respect to the axis 701 shown in FIG. 2) to slow or stop the spontaneous wicking of the sample into the fluid reservoir 812. While the plurality of projections 842 are generally trapezoidal in shape, the projections may be any geometrical shape (e.g., rectangle, oval, circle, etc.).

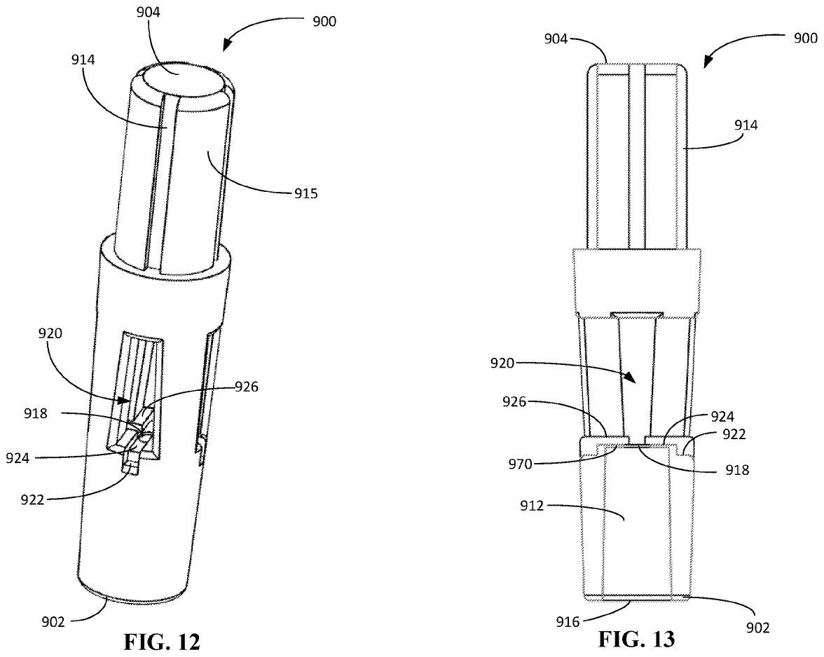

Referring now to FIGS. 12 and 13, another tip portion 900 is shown, according to an example embodiment. The tip portion 900 is configured to receive a predetermined volume of a sample (e.g., blood). For example, the tip portion 900 may be used to collect a sample for testing in the analyte detection system 100 described above. The tip portion 900 extends from a first end 902 to a second end 904 and may share one or more characteristics with the tip portion 800 discussed herein. For example, a projection 915 of the tip portion 900 may be received within the aperture 712 of the body portion 710 in a similar manner as the tip portion 800. Further, the tip portion 900 includes grooves 914 configured to receive projections 714 of the body portion 710 to prevent rotation of the tip portion 900 about the axis 701 (see FIG. 1) when the tip portion 900 is coupled to the body portion 710.

The tip portion includes a fluid reservoir 912 proximate the first end 902. The fluid reservoir 912 extends from a first aperture 916 to a second aperture 918 and is configured to receive a predetermined volume of a sample (e.g., blood). The fluid reservoir 912 may be the same, or similar to, the fluid reservoir 812 described above. For example, the second aperture 918 of the fluid reservoir 912 is in fluid connection with one or more lateral windows 920 that surround a portion of the tip portion 900.

As shown, each of the lateral windows 920 includes a first ledge 922 defined by a cut out in the tip portion 900. The first ledge 922 includes a flat portion that is positioned outside of the fluid reservoir 912. The first ledge 922 is further positioned at a first height that is above the first aperture 916 and below the second aperture 918. The first ledge 922 may facilitate air flow out of the second aperture 918 to facilitate spontaneous wicking of the sample in the fluid reservoir 912. Further, the first ledge 922 may interface with one or more components of the cartridge device 300 (e.g., for securing the tip portion 900 within the cartridge device 300).

Each of the lateral windows 920 includes a second ledge 924 defined by a cut out in the tip portion 900. The second ledge 924 is positioned at a second height that is flush with the top of the second aperture 918, thereby resulting in a relatively thin ledge 970 (e.g., rim) surrounding the second aperture 918. The relatively thin nature of the ledge 970 may result in improved visibility of the second aperture 918 by a user of the tip portion 900. For example, the ledge 970 and the second aperture 918 may operate as a stop to prevent or slow capillary action of the sample past the bottom portion of the ledge 970. Since the ledge 970 is relatively thin (e.g., as a result of the second ledge 924), a user's visibility of the sample within the fluid reservoir 912 may be improved. Further, the second ledge 924 may facilitate air flow out of the second aperture 918 to facilitate spontaneous wicking of the sample in the fluid reservoir 912.

Each of the lateral windows 920 includes a third ledge 926. The third ledge 926 is positioned at a third height that is above the first ledge 922 and the second ledge 924. The third ledge 926 may improve the structural strength and rigidity of the tip portion while still facilitating air flow out of the second aperture 918 to facilitate spontaneous wicking of the sample in the fluid reservoir 912.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

As used in the specification and claims, the singular form “a”, “an” and “the” include both singular and plural references unless the context clearly dictates otherwise. For example, the term “a molecule” may include, and is contemplated to include, a plurality of molecules. At times, the claims and disclosure may include terms such as “a plurality,” “one or more,” or “at least one;” however, the absence of such terms is not intended to mean, and should not be interpreted to mean, that a plurality is not conceived.

As used herein, the term “comprising” or “comprises” is intended to mean that the devices, systems, and methods include the recited elements, and may additionally include any other elements.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

What is claimed is:

1. A sample collection device for collecting a biological sample for analysis in a detection system, comprising:
a body portion extending along an axis between a first end to a second end opposite the first end; and
a tip portion between a third end and a fourth end opposite the third end, the fourth end being coupled to the body portion proximate the second end, the tip portion comprising:
a fluid reservoir extending into the tip portion proximate the third end; and
one or more lateral windows extending into the tip portion in a direction perpendicular to the axis such that a portion of the fluid reservoir is in fluid communication via the one or more lateral windows.

2. The sample collection device of claim 1, wherein the fluid reservoir extends substantially parallel to the axis from a first aperture proximate the third end to a second aperture.

3. The sample collection device of claim 2, wherein the tip portion includes a clear portion such that at least a portion of the fluid reservoir is visible via the clear portion.

4. The sample collection device of claim 2, wherein the first aperture defines a first cross-sectional area and the second aperture defines a second cross-sectional area that is smaller than the first cross-sectional area.

5. The sample collection device of claim 2, wherein the fluid reservoir includes a ledge surrounding a perimeter of the second aperture, the ledge including a plurality of projections surrounding the perimeter of the second aperture.

6. The sample collection device of claim 2, wherein the fluid reservoir includes the first aperture proximate the third end, the first aperture extending into the tip portion a first depth such that the fluid reservoir defines a cross section from the first aperture to the first depth, wherein the cross section is greater proximate the first aperture than proximate the second aperture.

7. The sample collection device of claim 6, wherein the first aperture is an oval shaped aperture defining a first axis and a second axis that is smaller than the first axis.

8. The sample collection device of claim 7, wherein the first axis is greater than 0.8 mm and less than 1.6 mm and wherein the second axis is between 0.6 mm and 1.4 mm.

9. The sample collection device of claim 8, wherein the first depth is between 1.7 mm and 2.5 mm.

10. The sample collection device of claim 1, wherein a portion of the fluid reservoir includes a hydrophilic coating.

11. The sample collection device of claim 1, wherein a portion of the fluid reservoir includes an anticoagulant coating.

12. The sample collection device of claim 2, wherein the one or more lateral windows includes:
a first trapezoidal window having a first base edge proximate the second aperture and a second base edge opposite the first base edge, the second base edge being longer than the first base edge; and
a second trapezoidal window having a third base edge proximate the second aperture and a fourth base edge opposite the first base edge, the third base edge being longer than the second base edge.

13. The sample collection device of claim 1, wherein the tip portion is removably coupled to the body portion.

14. A system, comprising:
a cartridge including a cartridge aperture; and
a sample collection device configured to provide a sample to an interior of the cartridge, the sample collection device including:
a tip portion configured to be at least partially received within the cartridge aperture, the tip portion including a tip body extending between a first tip end and second tip end opposite the first tip end, the tip portion comprising:
a fluid reservoir configured to receive the sample and positioned between a first tip aperture proximate the first tip end and a second tip aperture, the first tip aperture defining a first cross-sectional area and the second tip aperture defining a second cross-sectional area smaller than the first cross-sectional area; and
one or more windows extending into the sample collection device such that the second tip aperture is in fluid communication with the one or more windows.

15. The system of claim 14, wherein the tip portion includes one or more projections extending into the fluid reservoir proximate the second tip aperture, the one or more projections reducing a cross-sectional area of the fluid reservoir.

16. The system of claim 14, wherein the tip portion includes a ledge extending in a first direction through the tip body, the ledge positioned between the fluid reservoir and the one or more windows.

17. The system of claim 16, wherein the ledge is a first ledge and the tip portion includes a second ledge extending from the first ledge in a second direction transverse the first direction such that the second ledge extends beyond the second tip aperture.

18. A sample collection device configured to collect a sample, the sample collection device comprising:
sample collection body;
a tip portion coupled to the sample collection body, the tip portion including a fluid reservoir configured to receive the sample and positioned between a first tip aperture proximate a first tip end and a second tip aperture; and
one or more windows extending into the sample collection device such that the first tip aperture and the second tip aperture is in fluid communication with the one or more windows.

19. The sample collection device of claim 18, wherein the fluid reservoir extends along a first axis, and wherein a cross-sectional area defined by the fluid reservoir decreases along the first axis from the first tip aperture towards the second tip aperture.

20. The sample collection device of claim 19, wherein the tip portion includes one or more projections extending into the fluid reservoir proximate the second tip aperture, the one or more projections reducing the cross-sectional area of the fluid reservoir.
